(19) United States
(12) Patent Application Publication
(10) Pub. No.: US 2026/0020879 A1
(43) Pub. Date: Jan. 22, 2026

(54) SYSTEMS AND METHODS FOR PROVIDING A BIOLOGICAL SAMPLE COLLECTION CONTAINER AND LID ASSEMBLY

(71) Applicant: Kameron Ross Stilson, Imperial Beach, CA (US)

(72) Inventor: Kameron Ross Stilson, Imperial Beach, CA (US)

(21) Appl. No.: 19/275,799

(22) Filed: Jul. 21, 2025

Related U.S. Application Data
(60) Provisional application No. 63/673,584, filed on Jul. 19, 2024.

(51) Int. Cl. A61B 17/43

(52) U.S. Cl. CPC A61B 17/43

(57) ABSTRACT

The present disclosure relates generally to a sample collection container system, including: a container comprising a first opening; a sealing member coupled to the first opening of container, the sealing member comprising a second opening configured to receive a body part of an individual, wherein the second opening is smaller than the first opening, wherein the second opening is configured to form a seal against the body part of the individual during collection of a biofluid; and a lid configured to couple to the first opening of the container.

### 1. RELATED APPLICATION

This application claims priority to U.S. Provisional Patent Application Ser. No. 63/673,584 filed Jul. 19, 2024, entitled SAMPLE COLLECTION CONTAINER AND LID ASSEMBLY (Attorney Docket No. 36803.2); the entire disclosure of which is hereby incorporated by reference.

### BACKGROUND OF THE INVENTION

### 2. Field of the Invention

The present disclosure relates to sample collection container and lid systems, and more particularly to sample collection container systems that provide a seal around an anatomical site for production of a biological sample for collection.

### 3. Background

Biological samples such as semen, urine, saliva, and stools are often collected in sample containers or cups. Collected samples may be used for various purposes including diagnostic testing, monitoring health conditions, disease screening, medical research, and fertility treatments. Efficient and hygienic collection of biological samples may be difficult and/or uncomfortable for individuals due to the basic, cumbersome designs that are not ergonomically designed for human or animal anatomy. Problems with conventional sample collection containers and lid designs may allow for contamination of the biological sample, potentially rendering the sample ineffective for its purpose.

Thus, while techniques currently exist, challenges persist. Accordingly, it would be an improvement in the art to augment or even replace current techniques with other techniques and designs.

### BRIEF SUMMARY OF THE INVENTION

The present disclosure relates generally to a sample collection container system, including a container including a first opening; a sealing member coupled to the first opening of container, the sealing member including a second opening configured to receive a body part of an individual, wherein the second opening is smaller than the first opening, wherein the second opening is configured to form a seal against the body part of the individual during collection of a biofluid; and a lid configured to couple to the first opening of the container.

The present disclosure further relates generally to sample collection containers, including a container body configured to receive biofluid through a first opening; an intermediate lid coupled to the container body, the intermediate lid including a sealing member with a flexible, biocompatible material and a second opening configured to receive a body part of an individual, wherein the second opening of the sealing member is configured to form a seal against skin of the individual during collection of the biofluid; and a lid configured to couple to the intermediate lid securing the biofluid in the container body.

Moreover, the present disclosure relates generally to methods of obtaining biofluid samples from users through the use of a sample collection container system, the method including: providing a container including a first opening; coupling a sealing member to the first opening of the container, wherein the sealing member includes a second opening that is smaller than the first opening, wherein the second opening is configured to form a seal against a body part of a user; receiving the body part of the user through the second opening of the sealing member; receiving a biofluid sample; removing the body part of the user from the second opening of the sealing member; and without removing the sealing member, coupling a lid to the first opening of the container to seal the biofluid sample within the container.

While the methods and processes of the present invention have proven to be particularly useful in the area of collecting semen, those skilled in the art can appreciate that the methods and processes can be used to collect a variety of different biofluids.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only representative embodiments of the present invention and are, therefore, not to be considered limiting of its scope, embodiments of the present invention will be described and explained with additional specificity and detail through the use of the accompanying drawings in which:

FIGS. 1A-1B are illustrations of conventional containers;

FIG. 2 is an illustration depicting a representative container system, in accordance with one or more embodiments of the present disclosure;

FIGS. 3A-3C are illustrations depicting a representative container system with different sized penises, in accordance with one or more embodiments of the present disclosure;

FIGS. 4A-4B are illustrations depicting different functions of a representative container system, in accordance with one or more embodiments of the present disclosure;

FIGS. 5A-5B are illustrations showing a perspective and a side view of a representative container system, in accordance with one or more embodiments of the present disclosure;

FIG. 6A shows an exploded perspective view of a representative container system, in accordance with one or more embodiments of the present disclosure;

FIG. 6B shows a perspective view of a representative container and lid, in accordance with one or more embodiments of the present disclosure;

FIG. 7 shows a side cross-sectional view of a representative container system, in accordance with one or more embodiments of the present disclosure;

FIG. 8A shows a perspective cross-sectional view of a representative container system, in accordance with one or more embodiments of the present disclosure;

FIG. 8B shows a perspective view of a representative lid, in accordance with one or more embodiments of the present disclosure;

FIG. 8C shows a side cross-sectional view of a representative sealing member, in accordance with one or more embodiments of the present disclosure;

FIG. 9 shows a side view of a representative container, in accordance with one or more embodiments of the present disclosure;

FIGS. 10A-10C show various views of a representative container system, in accordance with one or more embodiments of the present disclosure; and

FIG. 11 shows an exploded view of a representative container system, in accordance with one or more embodiments of the present disclosure.

### DETAILED DESCRIPTION OF THE INVENTION

A description of embodiments of the present disclosure will now be given with reference to the Figures. It is expected that embodiments of the present disclosure may take many forms and shapes, hence the following disclosure is intended to be illustrative and not limiting. It is to be understood that the following detailed description is representative and explanatory and is not restrictive of embodiments of the invention, as claimed. It should be understood that the various embodiments are not limited to the arrangements and components shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

While there may be specific reference in this disclosure to semen collection during ejaculation, the disclosure is not limited to semen collection and may include a collection of a variety of other biological sample including urine, saliva, nasal discharge, vaginal discharge, stools, sweat, sputum, breast milk, or any other biofluid that is excreted or secreted from a body. Furthermore, the principles in this disclosure may be applied to animals as well as humans.

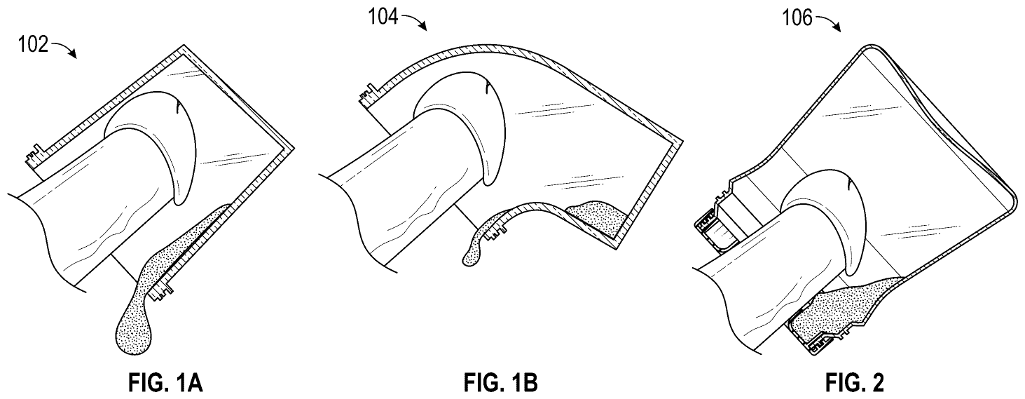

Sample containers (e.g., cups, jars, bottles, vials, tubes, bags, etc.) may be used for collection, storage of a sample, as well as for transportation of a sample from an individual's home or a physician's office to a laboratory for testing. In many cases, applications for the sample container include a repeatable and intimate process by the individual. For example, the individual may be a sperm donor that donates regularly at a sperm bank, or the individual may be going through one or more rounds of in vitro fertilization (IVF) or intrauterine insemination (IUI). Accordingly, it is important for individuals to feel at ease and comfortable. The method of masturbation and the angle of the penis upon ejaculation should allow for the greatest amount of semen to be collected, while maintaining integrity of the sample. Collection containers of the present disclosure improve upon conventional sample collection containers (e.g., containers 102 and 104 depicted in FIGS. 1A and 1B) by increasing control over the angle and ejection of the ejaculate by the individual. Additionally, leakage is avoided during collection, transit, or storage. Furthermore, in many instances, sample containers of the present disclosure provide additional user comfort. For example, the sample collection of the present disclosure avoids rough, sharp, and/or serrated edges, which, if present, could cause discomfort or pain for the individual when coming into contact with the sensitive skin of the penis. Additionally, contact between the penis and the sample collection container is likely, given the individual's goal of collecting the sample in the container without spilling or scattering any biofluid.

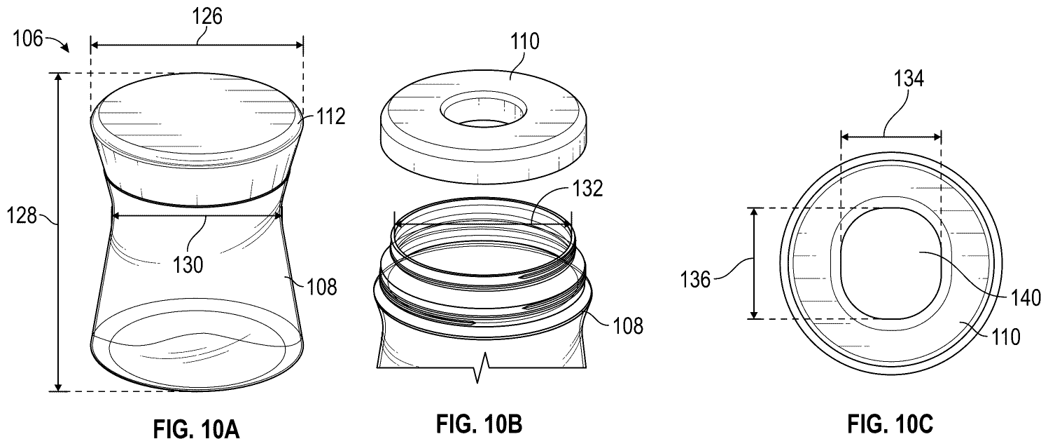

The collection container system 106 of the present disclosure corrects and improves any mismatched angles between a shaft of an individual's penis, the trajectory of ejaculate, and the required upright angle conventional sample containers have in order to collect and retain the sample (e.g., as shown in FIGS. 1A and 1B). In many conventional containers such as container 102 and/or 104, ejaculate can easily spill out and is not properly retained and sealed. The present disclosure solves the problem of this angle mismatch by sealing an opening 142 of container 108 and/or lid 112 with the penis such that the individual may align container 108 with the direction of the ejaculate while avoiding back flow or spillage (as illustrated with container system 106 in FIG. 2). In some embodiments, sealing of container 108 or lid 112 is accomplished by adding a sealing member 110 to the container system 106. In some cases, sealing member 110 includes a customized fit for a particular container 108. For example, in some embodiments, container 108 includes a sealing member 110 at the opening 142 of container 108 that allows for a distal end of a penis to be enclosed by sealing member 110 as semen is ejected from the urethral opening at the tip of the penis, thus preventing spills or leaks of the semen. In some embodiments, sealing member 110 is a part of lid 112 or a lid assembly 138 that is coupled to a sample collection container 108. In one or more embodiments, lid assembly 138 of the present disclosure includes multiple parts or portions (e.g., sealing member 110 and lid 112) that, in some embodiments, are coupled and/or fused together. In some embodiments, sealing member 110 and/or lid 112 have positive grip knurls surrounding a circumference of sealing member 110 and/or lid 112 that aid in coupling and de-coupling from the sample container 108 or from other parts of lid assembly 138. In some embodiments, the overall width of sealing member 110 and/or lid 112 does not exceed the width of the sample container 108 and other parts of lid assembly 138. In other embodiments, a width 126 of lid 112 exceeds a width 130 of a narrow portion of container 108, as shown in FIG. 10A.

In some embodiments, sealing member 110 includes one or more surfaces with beveled, chamfered, or rounded edges, especially in all areas where the skin of the penis may come in contact, eliminating any discomfort caused by rough, sharp, and/or serrated edges of conventional containers (e.g., conventional containers 102 and 104). In some embodiments, one or more portions of sealing member 110 comprises a flexible material (e.g., silicone or other material) and includes an opening 140 in sealing member 110. Sealing member 110 provides for a seal when the penis is pressed against and inserted through opening 140. In some embodiments (not shown), sealing member 110 includes a slit or pinhole that, when closed, seals the flexible layer. In such embodiments, the seal is broken by the penetration of the penis in the slit or pinhole of sealing member 110. In other embodiments, sealing member 110 is a septum or membrane, which includes opening 140 and/or a slit or pinhole. Sealing member 110 is generally comprised of silicone or any other material that: (i) provides the flexibility and strength to allow insertion of the glans of the penis into container 108 during ejaculation or urination; (ii) provides enough frictional force to support the weight of container 108 and the sample (e.g., ejaculate or urine) during and after ejaculation or urination; (iii) does not adversely affect the integrity of the sample itself, its accuracy in follow-on analyses, or its effectiveness in follow-on procedures; and (iv) ensures a perfect or near-perfect seal with container 108 and an adequate seal with skin of the penis during use by the individual.

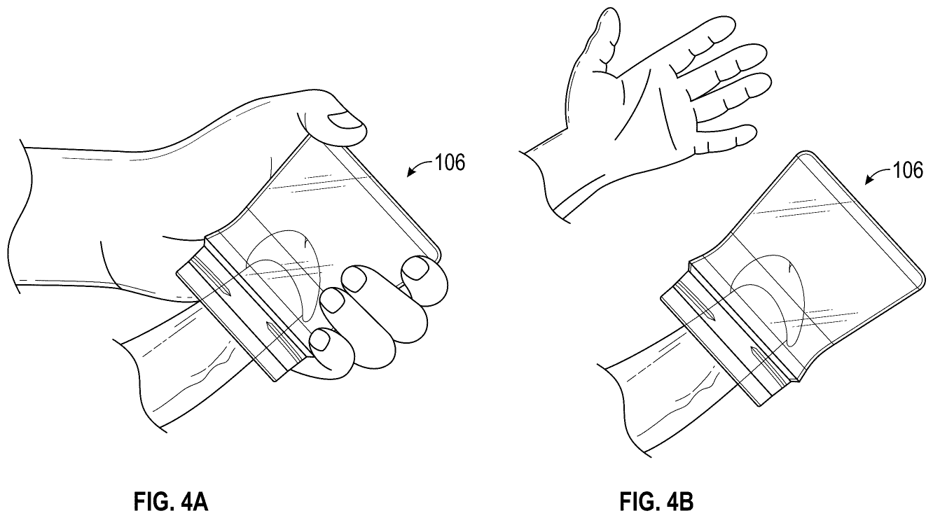

In one or more embodiments, an individual holds container system 106 with their hand while a sample is collected, as shown in FIG. 4A. In some embodiments, container system 106 is ergonomically shaped and designed to be easily hand-held. In some embodiments, the disclosed sample container system 106 is light enough that the frictional force between the skin of the penis and sealing member 110 is enough to support the weight of the container throughout the ejaculation process. For example, as shown in FIG. 4B, an individual can use container system 106 to collect a sample without holding container system 106 with their hands. Because this frictional force is enough to support the weight of the container, it is also enough to provide a seal between the container and lid assembly and the penis during ejaculation, eliminating any spills, scattering, or leakage of any biofluid, including in the event that the individual loses grip on the container system 106. The seal provided by the flexible layer also serves to remove excess fluid from the glans of the penis (e.g., when removing the penis from contact with sealing member 110), assisting in maintaining sample integrity within the sample container, minimizing the risk of contamination, and maximizing the volume of the collected sample fluid. Sealing member 110 allows for ejaculation to happen at a natural angle for the individual, without having to worry about missing the container or spilling any semen. In some embodiments, sealing member 110 is a component of an overall sample container system 106 including a sample container 108 and lid assembly 138 that includes sealing member 110 and lid 112 that are designed to be compatible only with each other. In other embodiments, sealing member 110 and/or lid assembly 138 is a standalone item that is configured to fit any standard sample collection containers. Furthermore, the sample container 108 including the flexible layer of material is configured to be compatible with standard lid designs.

In some embodiments, lid 112 is coupled to the top of container 108 to seal in the contents of the sample collection container for transport and/or storage following collection. In some embodiments, sealing member 110 can be discarded after the sample has been collected in container 108, in which case lid 112 couples to the sample container 108 itself for storage and transport. In some embodiments, sealing member 110 includes threads to receive lid 112 by being screwed together. In such embodiments, the threads are the same size as those of the container itself, which allows for lid 112 to couple to sealing member 110 or to the container 108 itself. While threads are mentioned in the disclosure and shown in at least some of the figures, all separable pieces of the sample container system 106 can be coupled and decoupled in any manner that maintains a seal between components that prevents leakage. For example, in some embodiments, lid 112 couples together with sealing member 110 or the sample container 108 with a snap fit or an interference fit. In other embodiments, lid 112 is vacuum sealed and/or includes push buttons, flaps, slides, or locks. In some embodiments, lid 112 has an annular protrusion 122 in the center of the container-facing side of lid 112 that is dimensioned such that it fits into and seals the opening 140 or the slit in the center of sealing member 110 and provides an additional seal during transportation and storage, preventing leakage and keeping more of the usable sample within container 108.

All materials used in the sample container system 106 are sterile and free of contaminants, according to industry standards, and to be made of materials that have no adverse effect on the integrity of the sample material and the accuracy of the follow-on analyses or procedures. For example, sample container 108 is of any type, scale, indication, transparency, or include other such qualities the collection, transportation, storage, and analysis process may require, or none of them.

FIG. 2 is an illustration that depicts how a sample container system 106 of the present disclosure fits and functions on an erect penis during ejaculation. In FIG. 2, an erect penis is pointed at an upward angle. The ejaculate may enter the sample container at an upward (i.e., expected) angle, but is prevented from spilling by the seal created around the distal end of the penis. The sample container system 106 is shown as fitting onto the erect penis during ejaculation, with the sealing member (e.g., sealing member 110) fitting snugly over the glans of the penis, providing both a seal that prevents any backflow or leakage as well as enough force to support the weight of the sample container from the frictional forces between the flexible layer and the skin of the penis. The sample container with the coupled lid assembly therefore provides an appropriate angle to match the trajectory of the biofluid without spilling, scattering, missing, or leaking the biofluid sample material. The sample container and coupled lid assembly avoid the need for the user to “aim” the ejaculate into the sample container while attempting to avoid spilling the ejaculate.

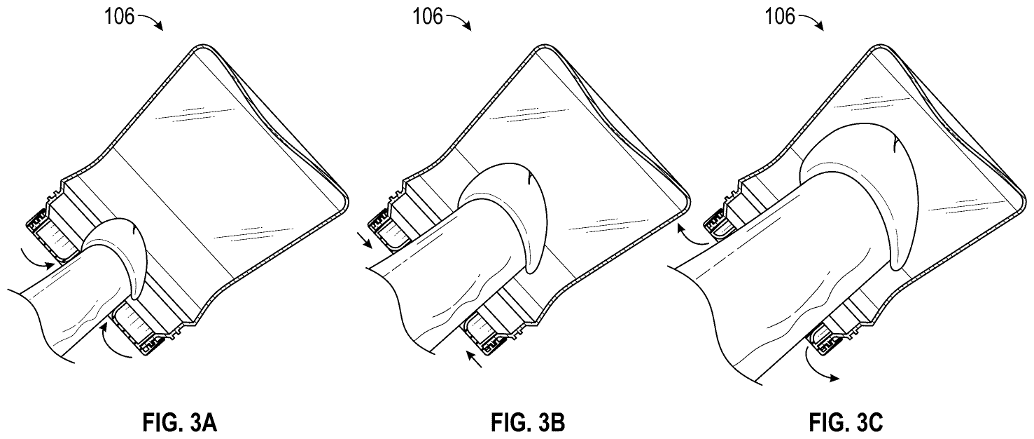

FIGS. 3A-3C are illustrations depicting a representative container and lid assembly showing the fit of different sized penises, in accordance with one or more embodiments of the present disclosure. Approximately 90% of males fall within the middle 50 percentile for erect penis diameter. Because sealing member 110 is elastic, flexible, and stretchable, opening 140 is small enough to fit smaller penises, and stretches enough to fit nearly all men, including extreme outliers. For example, as shown in FIG. 3A, container system 106 is configured such that a user with a penis size in the 5th percentile can still use container system 106 for collection of a biofluid sample. As shown in FIG. 3B, container system 106 is configured such that a user with a penis size in the 50th percentile can use container system 106 comfortably for collection of a biofluid sample. And as shown in FIG. 3C, container system 106 is configured such that a user with a penis size in the 95th percentile can use container system 106.

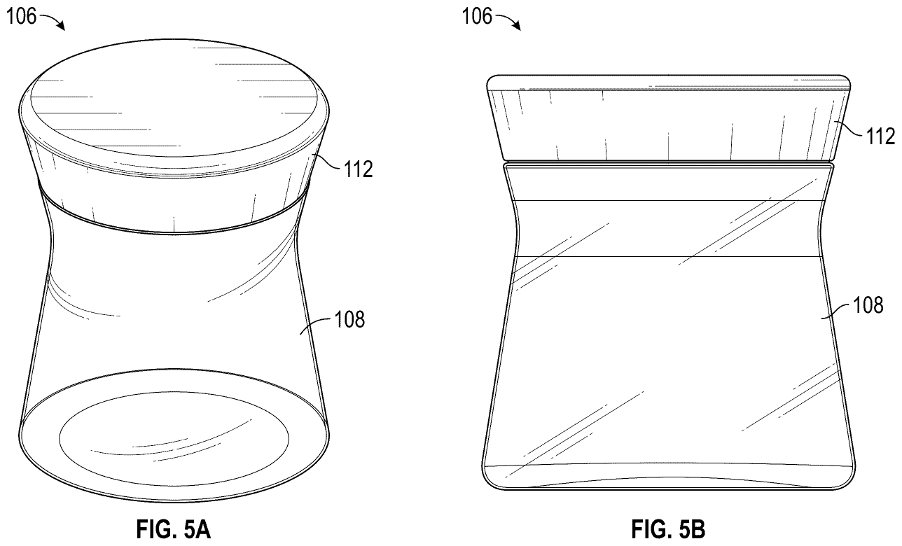

FIGS. 5A-5B are illustrations including a perspective and a side view of a representative container system 106, in accordance with one or more embodiments of the present disclosure. In the illustrated embodiment, the plastic of sample container 108 is sufficiently clear (e.g., transparent, or translucent) to see the sample inside sample container 108. In some embodiments, the interior texture of container 108 is a hi-gloss polished plastic, while the exterior texture is frosted plastic. In some embodiments, sample container 108 includes a label or writeable surface to identify the source of the sample, the date of sample collection, or other relevant information such as an identification/serial number. Sample container 108 is manufactured to prevent brittleness and ensure sufficient strength to maintain the seal during collection, transport, and storage. In some embodiments, sample container 108 is designed such that it may be stacked and nested with other sample containers to conserve pre-collection storage space. All materials of sample container system 106 are sterilized to prevent contamination of the sample as well as potential transmission of pathogens to the individual who collects the biofluid and all those who handle sample container 108 following the collection. In some embodiments, sample container 108 displays volumetric measurements on its side walls to assist with collection to ensure a proper amount of biofluid is collected.

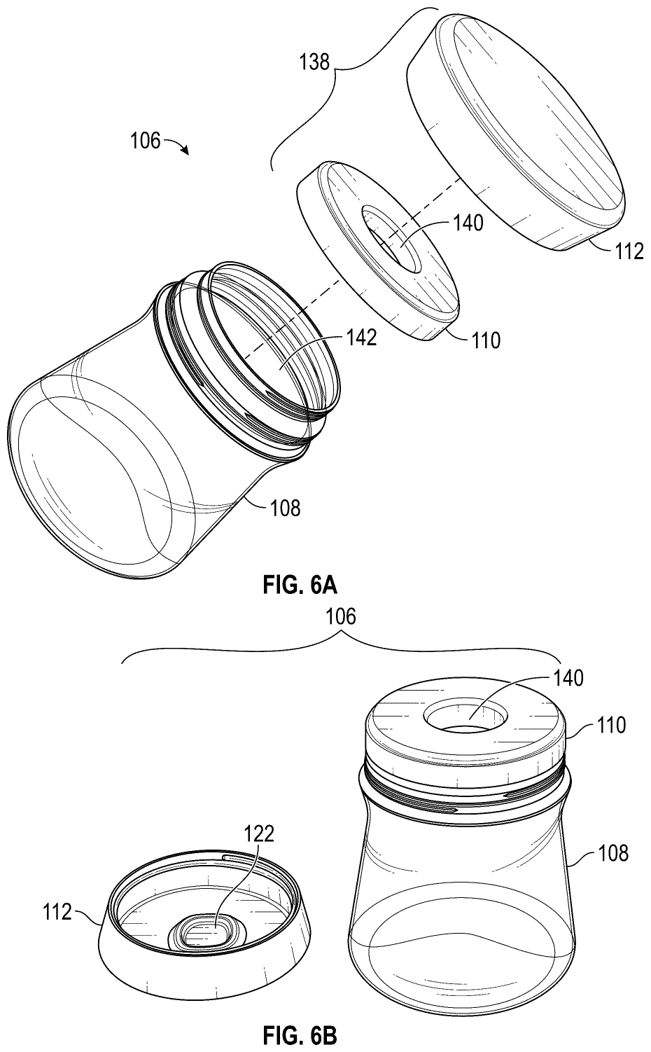

FIG. 6A is an illustration of an exploded view of container system 106, in accordance with one or more embodiments of the present disclosure. FIG. 6B is a perspective view of container system 106 with lid 112 detached from container 108 and sealing member 110 which are coupled together, in accordance with one or more embodiments of the present disclosure. As shown in FIG. 6B, sealing member 110 selectively couples to container 108 for a standard collection configuration, where a user can insert their penis into opening 140 and ejaculate or urinate to collect the biofluid sample. While FIGS. 6A and 6B show that sealing member 110 is separate and selectively de-couples from container 108, in some embodiments, sealing member 110 is integrally formed with container 108 as a monolithic unit. In other embodiments, while not integrally formed, sealing member 110 is permanently coupled to container 108 such that they form a monolithic unit following the permanent coupling.

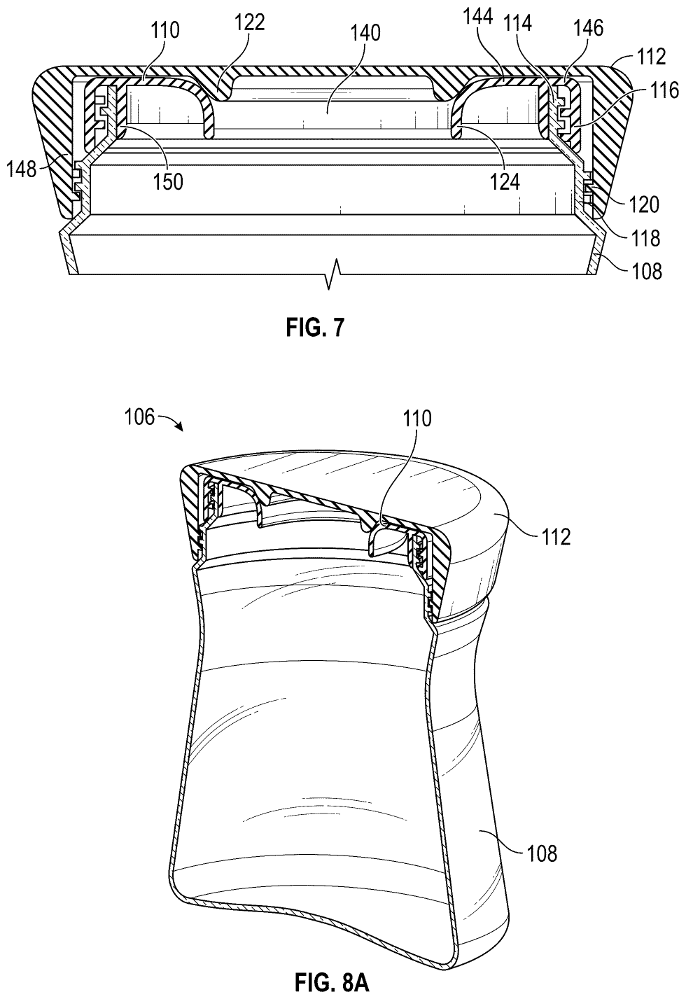

FIG. 7 shows an illustration of a cross-sectional view of the coupling of sealing member 110, lid 112, and container 108. In one or more embodiments, sealing member 110 and container 108 may be coupled in any suitable manner to perform the function of allowing a user to produce a sample within container 108 and sealing any produced fluid within container 108 to prevent leaks or spills. Additionally, in one or more embodiments, lid 112 is coupled to container 108 in any suitable manner to accomplish its functions of sealing the produced sample within container 108 and preventing leaks or spills. In one or more embodiments, container 108 includes two sets of stepped threads, including base threads 118 and upper threads 114, while sealing member 110 includes sealing member threads 116 and lid 112 includes lid threads 120. In some cases, base threads 118 are configured to correspond to and couple or mate with lid threads 120 to couple or decouple lid 112 to and from container 108. In some instances, upper threads 114 are configured to correspond to and couple or mate with sealing member threads 116 to couple or decouple sealing member 110 to and from container 108. In some embodiments, any of base threads 118, upper threads 114, lid threads 120, or sealing member threads 116 are integrated with silicone or other sealing agent that ensures a leak-free seal when tightened. In some cases, one or more gaskets or O-rings may be used to further seal the contents of container 108.

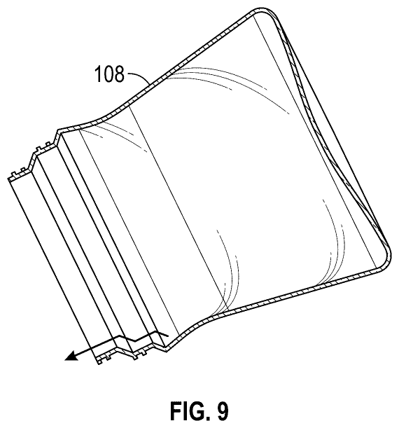

In some embodiments, the circumference of base threads 118 is greater than the circumference of upper threads 114. This allows for both sealing member 110 and lid 112 to couple to container 108 at the same time. In one or more embodiments, an outer diameter of sealing member 110 is less than an inner diameter of lid 112, which allows lid 112 to fit over and surround sealing member 110 when coupled to container 108. In this way, each of sealing member 110 and lid 112 couples to container 108 at the same time. Additionally, each of sealing member 110 and lid 112 can be coupled independently to container 108 with or without the other. The transition portions of container 108 between base threads 118 and upper threads 114 may be angled such that any collected sample can flow easily out of container 108 (as shown in FIG. 9). For example, the transition between base threads 118 and upper threads are obtuse angles such that none of the sample pools in right or acute angled transition sections between the stepped threads.

In one or more embodiments, lid 112 includes an outer wall 148 that may be generally cylindrical and includes additional contours for ergonomics. In some embodiments, a center of lid 112 includes annular protrusion 122 that extends downward from the top cover portion of lid 112 and aligns with opening 140 to seal and prevent fluid from entering any cracks formed between lid 112, sealing member 110, and container 108. In some embodiments, lid 112 includes a solid plug that aligns with and contacts opening 140 to seal and prevent fluid leakage. While portions of sealing member 110 are generally flexible, lid 112 is generally inflexible.

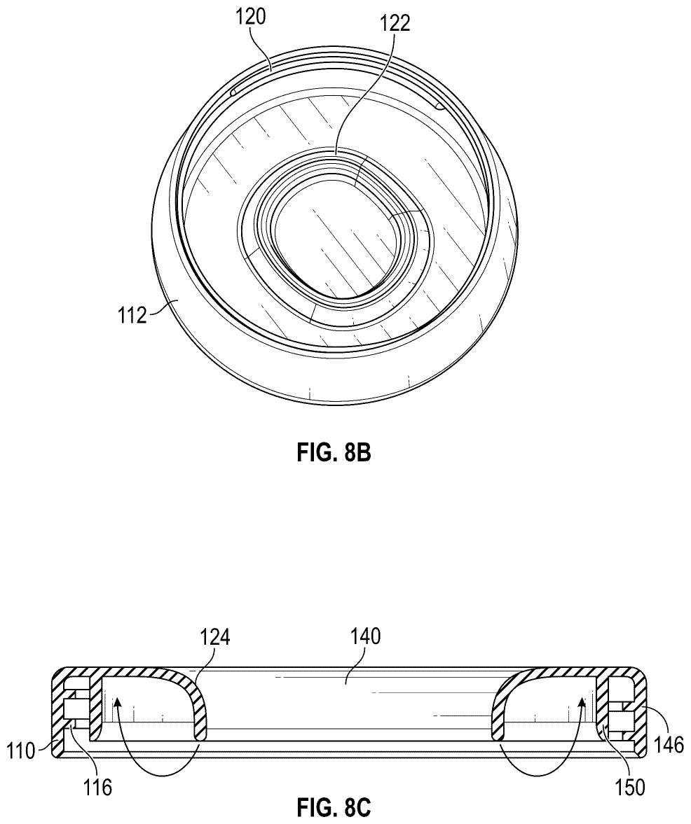

In one or more embodiments, sealing member 110 includes a generally planar cover portion 144 with an outer wall 146, an inner wall 150, and an inner annular flange 124 that each extend downward. Outer wall 146 includes internally facing sealing member threads 116. Inner wall 150 extends downwardly opposite outer wall 146 with the top lip of container 108 that includes upper threads 114 is disposed between outer wall 146 and inner wall 150. Inner wall 150 adds structural stability to sealing member 110 and prevents annular flange 124 from collapsing. In some embodiments, annular flange 124 surrounds and defines opening 140 and is configured to gradually extend downward, which avoids any sharp edges for the sensitive skin of a user. In some embodiments, annular flange 124 can flex such that as a penis is inserted through opening 140, annular flange 124 is pushed towards inner wall 150, expanding opening 140 and allowing for a snug seal (as shown in FIG. 8C).

FIGS. 10A-10C show various dimensions associated with container system 106, in accordance with one or more embodiments of the present disclosure. Container system 106 can have any suitable height that allows it to function as described herein. For example, in some instances container system 106 (i.e., container 108 with lid 112 attached) has a height 128 of between 50 millimeters and 120 millimeters, or within any suitable subrange therein. For example, in one or more embodiments, height 128 is between 70 millimeters and 100 millimeters, or between 80 mm and 90 mm. Indeed, in some embodiments, height 128 is between 84 millimeters and 85 millimeters, and in some embodiments height 128 is 84.7 millimeters.

Container system 106 can have varying widths that allow it to function as described herein. For example, in some instances, container system 106 (i.e., container 108) has a minimum width 130 of between 40 millimeters and 100 millimeters, or within any suitable subrange therein. For example, in one or more embodiments, width 130 is between 50 millimeters and 90 millimeters, or between 65 millimeters and 80 millimeters. Indeed, in some embodiments, width 130 is between 70 millimeters and 75 millimeters, and in some embodiments, width 130 is 73 millimeters. In some instances, container system 106 (container 108 and/or lid 112) has a maximum width 126 of between 50 millimeters and 150 millimeters, or within any suitable subrange therein. For example, in some embodiments, width 126 is between 75 millimeters and 125 millimeters or between 90 millimeters and 100 millimeters. Indeed, in some embodiments, width 126 is 95 millimeters.

Container 108 can have any suitable width 132 at its top lip portion that allows container 108 to perform its functions. For example, in some instances, the top lip portion of container 108 has a width 132 of between 20 millimeters and 80 millimeters, or within any suitable subrange therein. For example, in one or more embodiments, width 132 is between 30 millimeters and 70 millimeters or between 40 millimeters and 60 millimeters. Indeed, in some cases, width 132 is between 50 millimeters and 55 millimeters, and in some instances, width 132 is 53 millimeters.

Sealing member 110 may include various dimensions that allow it to function and serve its purposes as described herein. In some cases, opening 140 is not circular, but is a rounded rectangle or stadium shape with one side being longer than the other. Therefore, in some cases, opening 140 has a length 136 and a width 134, where length 136 is longer than width 134. Length 136 and width 134 can have any suitable dimensions that allow opening 140 of sealing member 110 to perform its functions as described herein. For example, in some cases, opening 140 can have a length 136 of between 10 millimeters and 40 millimeters, or any suitable subrange therein. In some embodiments, length 136 is between 15 millimeters and 35 millimeters or between 20 millimeters and 30 millimeters. Indeed, in some embodiments, length 136 is 24 millimeters. In some cases, opening 140 can have a width 134 of between 10 millimeters and 30 millimeters, or any suitable subrange therein. In some embodiments, width 134 is between 15 millimeters and 25 millimeters or between 20 millimeters and 23 millimeters. Indeed, in some embodiments, width 134 is 21 millimeters.

The components of container system 106 (i.e., lid 112, sealing member 110, and container 108) can be manufactured using any suitable technique. For example, in one or more embodiments, lid 112 is manufactured using injection molding. As another example, in some embodiments, sealing member 110 is manufactured using compression molding. By way of further example, in one or more embodiments, container 108 is manufactured using blow molding.

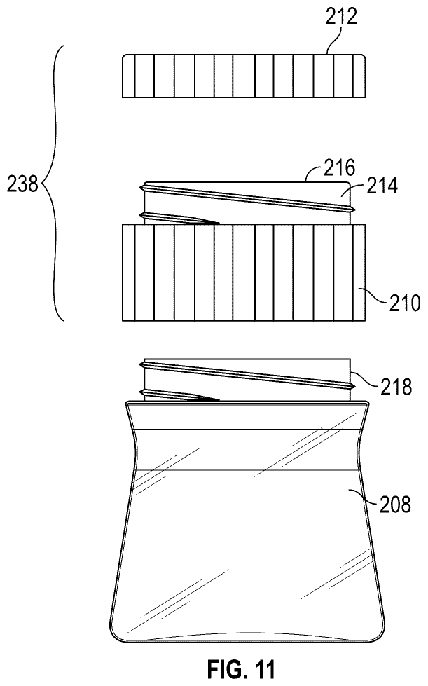

FIG. 11 shows an exploded side view of an alternative embodiment of a representative container 208 and lid assembly 238, in accordance with one or more embodiments of the present disclosure. In one or more embodiments, a sample container 208 is a cylindrical container with a narrower cylindrical top portion 218 with external threads that are configured to receive an intermediate lid 210 with corresponding internal threads. In some embodiments, container 208 does not have any threads but is instead configured to receive a snap-style lid. In such embodiments, sample container 208 includes a flange (not shown) that extends outward that receives a flexible lid that snaps around the flange, ensuring a proper seal of the biological sample collected.

In some embodiments, sample container 208 is a standard sample collection container (e.g., cup or jar) including a container body and an opening at the top of the container body, wherein a lid and/or lid assembly is customized in accordance with this disclosure. However, in other embodiments, elements of the lid and/or lid assembly are incorporated and/or integrated with container 208 such that intermediate lid 210 or top lid 212 are examples of a standard lid that screws or snaps onto a modified container in accordance with this disclosure. For example, FIG. 11 illustrates a representative embodiment where the lid assembly 238 is enhanced with intermediate lid 210, a beveled edge portion 214 that rises out of intermediate lid 210, a flexible layer 216 (e.g., a sealing member) at the top of beveled edge portion 214 of the intermediate lid 210 with a small hole or opening in the center of flexible layer 216 (e.g., a silicone layer), and a top lid 212. However, some embodiments combine intermediate lid 210 and sample container 208 such that, instead of intermediate lid 210, sample container 208 has beveled edge portion 214 rising out of the top thereof with a flexible layer and a small hole or opening in the center the of the flexible layer. In yet other embodiments, flexible layer 216 is flush with, or below, the top of sample container 208 with no beveled edge rising out of the sample container.

In some embodiments, intermediate lid 210, shown in FIG. 11 from a side view, couples to sample container 208 (e.g., to the opening of the body of sample container 208) during and after sample extraction and seamlessly supports beveled edge portion 214 that rises out of intermediate lid 210. In some embodiments, intermediate lid 210 includes dual threaded fasteners to help ensure a proper seal between intermediate lid 210 and the sample container. In some embodiments, intermediate lid 210 includes grip knurls around its circumference to assist in coupling and decoupling intermediate lid 210 with the sample container or the top lid.

Beveled edge portion 214 that rises out of intermediate lid 210 includes a hard plastic cylindrical portion that is narrower than intermediate lid 210. In some embodiments, beveled edge portion 214 transitions into a beveled or chamfered edge near the top of beveled edge portion 214. The top of beveled edge portion 214 houses flexible layer 216 (e.g., silicone ring) that will receive the penis during collection. In one or more embodiments, flexible layer 216 is seamlessly fused and/or integrated with the hard plastic of beveled edge portion 214. The hard plastic is smooth and any rough or sharp edges are removed during manufacture. The hard plastic portion of beveled edge portion 214 includes external dual threads that are configured to receive top lid 212 over flexible layer 216. In some embodiments, the entire beveled edge portion 214 rising out of intermediate lid 210 is silicone or some other flexible material instead of hard plastic. In such embodiments, intermediate lid 210 includes external or internal threads configured to receive top lid 212 instead of the beveled edge portion 214 of intermediate lid 210.

Flexible layer 216 or sealing member made of a material (e.g., a biocompatible material) such as silicone is at the top of beveled edge portion 214 of intermediate lid 210. The disclosure is not limited to silicone but may include any flexible material that provides a flexible seal between the skin of the penis and the lid/sample container to prevent any backflow or leakage. In some embodiments, there is an opening or hole in the middle of the flexible layer. In some embodiments, the opening or hole is circular while in other embodiments, the opening is a slit or other shape. The hole is used as a penis insertion point to fit over the glans tight enough to provide a seal against the skin of the penis to prevent any backflow. Backflow is a possibility since the sample container and lid assembly of the present disclosure allow the individual to place the container and lid assembly on the penis at any angle (e.g., upside down or mostly upside down) for collection of semen or urine. In some embodiments, flexible layer 216 includes a slit or pinhole that is flexible enough to expand and allow the penis to be inserted through the flexible layer. In some embodiments, flexible layer 216 is flush with or below the top of intermediate lid 210, avoiding the need for beveled edge portion 214 of intermediate lid 210.

In some embodiments, top lid 212 is coupled over beveled edge portion 214 to the top of intermediate lid 210 to seal in the collected sample. The sample is sealed with an air-tight seal due to a gasket (e.g., a rubber gasket) in top lid. In one or more embodiments, top lid 212 includes a protrusion in the center of the inside of top lid 212 (not shown in FIG. 11) that extends downward. The protrusion may be circular (e.g., cylindrical or annular), made of plastic (e.g., hard plastic) or a flexible sealing material, and fits snugly within the hole in the middle of flexible layer 216. The protrusion provides a seal with flexible layer 216 to prevent leakage during transport and storage. In some embodiments, top lid 212 includes grip knurls around its circumference to assist in coupling and decoupling of top lid 212.

In some embodiments, the lid assembly 238 includes intermediate lid 210, beveled edge portion 214 of intermediate lid 210, flexible layer 216, and top lid 212. In one or mor embodiments, lid assembly 238 is configured to provide a seal between an individual's skin and sample container 208 during ejaculation or urination. Lid assembly 238 is also configured to seal the contents of sample container 208 when top lid 212 is coupled to intermediate lid 210 and/or sample container 208. Such a seal prevents leakage of the collected sample in sample container 208 due to any pressure differentials that are experienced during shipping, transit, and/or storage. In one or more embodiments, lid assembly 238 is stackable and nestable to conserve pre-collection storage space. In some embodiments, lid assembly 238 is configured such that a sample container may be stably stacked on top of the lid assembly. In some embodiments, the lid assembly 238 is designed to fit any standard sample container that is already produced.

The present invention may be embodied in other specific forms without departing from its spirit or essential characteristics. The described embodiments are to be considered in all respects only as illustrative and not restrictive.

What is claimed and desired to be secured by Letters Patent is:

1. A sample collection container system, comprising:
a container comprising a first opening;
a sealing member coupled to the first opening of container, the sealing member comprising a second opening configured to receive a body part of an individual, wherein the second opening is smaller than the first opening, wherein the second opening is configured to form a seal against the body part of the individual during collection of a biofluid; and
a lid configured to couple to the first opening of the container.

2. The sample collection container system of claim 1, wherein the sealing member is disposed substantially between the container and the lid.

3. The sample collection container system of claim 1, wherein the container comprises a first set of threads and a second set of threads, wherein the sealing member is configured to couple to the container via the first set of threads, and wherein the lid is configured to couple to the container via the second set of threads.

4. The sample collection container system of claim 3, wherein the first set of threads has a smaller circumference than the second set of threads.

5. The sample collection container system of claim 1, wherein the lid comprises an annular protrusion that extends downward to contact and seal the second opening of the sealing member.

6. The sample collection container system of claim 1, wherein the body part can support the weight of the sample collection container system without the use of hands.

7. The sample collection container system of claim 1, wherein the lid comprises an outer wall that extends downward, wherein an outer diameter of the sealing member is less than an inner diameter of the outer wall.

8. The sample collection container system of claim 1, wherein the second opening of the sealing member is defined by an annular flange that extends downward.

9. The sample collection container system of claim 8, wherein the annular flange is flexible such that the second opening expands as the body part is introduced through the second opening.

10. The sample collection container system of claim 1, wherein the sealing member comprises silicone.

11. The sample collection container system of claim 1, wherein the sealing member comprises an outer wall and an inner wall, where the outer wall is disposed outside a top lip portion of the container, and the inner wall is disposed inside the top lip portion of the container.

12. The sample collection container system of claim 11, wherein the outer wall comprises threads that are configured to mate with external threads of the container.

13. A sample collection container, comprising:
a container body configured to receive biofluid through a first opening;
an intermediate lid coupled to the container body, the intermediate lid comprising a sealing member with a flexible, biocompatible material and a second opening configured to receive a body part of an individual, wherein the second opening of the sealing member is configured to form a seal against skin of the individual during collection of the biofluid; and
a lid configured to couple to the intermediate lid securing the biofluid in the container body.

14. The sample collection container of claim 1, wherein the second opening is sized to receive a penis of the individual.

15. The sample collection container of claim 1, wherein the second opening is a slit.

16. The sample collection container of claim 1, wherein the sealing member is integrally formed with the intermediate lid as a monolithic unit.

17. The sample collection container of claim 1, wherein the intermediate lid is integrally formed with the container body as a monolithic unit.

18. The sample collection container of claim 1, wherein the intermediate lid comprises:
a substantially cylindrical portion configured to couple to the container body; and
a top intermediate lid portion configured to couple with the lid,
wherein the sealing member is integrated with the top intermediate lid portion.

19. A method of obtaining a biofluid sample from a user using a sample collection container system, the method comprising:
providing a container comprising a first opening;
coupling a sealing member to the first opening of the container, wherein the sealing member comprises a second opening that is smaller than the first opening, wherein the second opening is configured to form a seal against a body part of a user;
receiving the body part of the user through the second opening of the sealing member;
receiving a biofluid sample;
removing the body part of the user from the second opening of the sealing member; and
without removing the sealing member, coupling a lid to the first opening of the container to seal the biofluid sample within the container.

20. The method of claim 19, wherein the lid comprises an annular protrusion that contacts and seals the second opening to prevent leakage.
